(19) United States
(12) Patent Application Publication
(10) Pub. No.: US 2005/0244393 A1
(43) Pub. Date: Nov. 3, 2005

(54) Sealant or tissue generating product

(75) Inventors: Pierre Philippart, Brussels (BE); Michele Brasseur, Brussels (BE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: Henogen S.A., Charleroi (BE)

(21) Appl. No.: 10/516,676

(22) PCT Filed: Jun. 20, 2003
(86) PCT No.: PCT/IB03/03245

§ 371 Date: Apr. 25, 2005

Related U.S. Application Data
(63) Continuation-in-part of application No. 10/176,401, filed on Jun. 21, 2002.
Application No. 10/176,401 is a continuation-in-part of application No. 09/469,302, filed on Dec. 22, 1999, now abandoned.

(30) Foreign Application Priority Data
Dec. 20, 2000 (WO) PCT/BE00/00152
Aug. 16, 2000 (WO) PCT/BE00/00094

(51) Int. Cl. A61K 45/00

(52) U.S. Cl. 424/93.72

(57) ABSTRACT

The present invention is related to a sealant or a tisuue generating product comprising a (coagulated) plasma matrix, one or more growth factors, at least one phospholipid and a protein scatffold for the generation of said tissue (or the coagulation factor VII).

### FIELD OF THE INVENTION

The present invention relates to a sealant or a tissue (a hard tissue such as bone or cartilage, or a soft tissue, such as skin or the epithelial tissue of the stomach) generating product comprising a (coagulated) plasma matrix, one or more growth factor(s), at least one phospholipid and a protein scatffold for the generation of said tissue (or the coagulation factor VII).

### BACKGROUND OF THE INVENTION AND STATE OF THE ART

Many researches have been made for the preparation of tissues substitute or implant.

For instance, for the preparation of bone substitute or implant, it is known to treat human bone by chemicals for destroying prions. The so treated human bone acts as a porous matrix suitable for the growth of cells after its implant.

It has also been proposed to prepare artificial matrix or sponge from collagen containing material and to use said matrix or sponge as bone substitute.

Example 4 of U.S. Pat. No. 5,733,545 discloses the preparation of clot from a mixture containing a plasma-buffy coat concentrate and ground dry bone or from a plasma-buffy coat concentrate and CaCl₂, said latter compound being used for ensuring the coagulation of the mixture. In said example 4, it is stated that the chelation of the plasma-buffy coat concentrate containing ground dry bone is possibly due to the presence of calcium from the solid bone. In said example, it is clearly stipulated that the use of thrombin is a cause of patient complications.

However, the bone substitute obtained by mixing a plasma-buffy coat concentrate and ground dry bone was not suitable for the bone generation.

For inducing bone repair, Friadent is commercializing the product “PEPGEN 15” and “pepgen 15 Flow” which consist of a synthetic resorbable matrix containing a synthetic amino acid peptide of formula GTPGPQGIAGQRGVV (PEPGEN15), and inorganic particles containing calcium phosphate and having a mean particle size lower than 750 μm.

The following table gives the correlation between the amino acid, its one-letter code and its three letter code.

| | One-letter | Three-letter |
|---|---|---|
| Amino Acid | Symbol | Symbol |
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Gln |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

This product enables good histologic regeneration in 2/4 cases evaluated.

### SUMMARY OF THE INVENTION

The present invention is related to a tissue generating product comprising a (coagulated) plasma matrix, (preferably a (coagulated) matrix of platelet rich plasma, or a (coagulated) matrix of platelet poor plasma), one or more growth factor(s)), preferably a recombinant compound for generating thrombin, at least one phospholipid and a protein scatffold for the generation of said tissue.

The protein scatffold present in the tissue generating product according to the invention is chosen according to the type of tissue which should be regenerated. This tissue could be a hard tissue (such as a bone or cartilage) or a soft tissue (such as skin or an epithelial tissue of the stomach). Preferably, the tissue generating product according to the invention is a bone generating product which comprises as a protein scatfold for the generation of said tissue a bio-engineerated osteo inductive bone substitute, such as the compound PEPGEN P15™ or PEPGEN P15 Flow (sold by the Company CERAMED) and which comprises an inorganic bovine bone material (as said protein scatfold for the generation of said bone tissue) and a peptide of 15 amino acid obtained from collagen type I involved in adhesion of reparative cell and having the formula GTPGPQGIAGQRGVV.

Another example of such bio-engineerated osteo inductive bone substitute is the compound OSIGRAFT™, (sold by the Company STRIKER) which is a pure resorbable protein scatffold obtained from bovine collagen comprising Heptotermin a (recombinant osteogenic protein 1), which is a bone morphogenetic protein 7 produced in CHO cells and which initiates bone formation through induction of cellular differentiation in mesenchymal cells.

Another example of such bio-engineerated osteo inductive bone substitute is the compound Ossigel™ (sold by the Company ORQUEST) which comprises the compound Hyaluronic acid which is a viscoelastic polymer complement to the activity of the tissue factor bFGF (basis fibroblast growth factor) which is advantageously a mytogen and potent angiogenic factor.

A further example of such bio-engineerated osteo inductive bone substitute is the compound Infuse™ (sold by the Company MEDTRONIC) which is a collagen sponge and which comprises a recombinant human bone morphogenetic 2 (rhBMP2) which could be used for the stimulation of new bone formation.

A last example of said protein scatffold is a matrix of collagen, reticulin and/or elastin fibers or their precursors (tropocollagen, tropoelastin, . . . ).

The protein scatffold for the bone tissue generation used in the tissue generating product according to the invention is an artificial matrix or sponge which further comprises one or more tissue factors, as well as other elements that facilitate the tissue regeneration, such as effective amount of calcium containing compound used for regeneration of a bone tissue.

Advantageously, the tissue generating product according to the invention comprises a matrix of coagulated platelet rich plasma with a high concentration of platelets (GERNOT WEIBRICH et al, Clin. Otal. Impl. Res. 13, p. 437-443, 2002; GERNOT WEIBRICH et al, Clin. Otal. Impl. Res. 14, p. 357-362, 2003). The platelet concentration (comprising different growth factors) will advantageously present an improved influence upon the regeneration of the tissue.

Advantageously, a coagulated matrix of platelet rich plasma has a platelet concentration higher than 1,500,000 platelets per microlitre of the matrix forming agents.

Preferably, said platelet rich plasma has a platelet concentration comprised between 1,500,000 platelets and 20,000,000 platelets per microlitre of the matrix forming agents.

Preferably, said concentration is comprised between 2,000,000 platelets per microlitre and 8,000,000 platelets per microlitre of the matrix forming agents, more preferably about 5,000,000 platelets per microlitre.

The tissue generating product may also comprise a coagulated matrix of platelet poor plasma, which means that the platelet concentration is advantageously lower than 1,500,000 platelets per microlitre of the matrix forming agents, preferably the platelet concentration is lower 500,000, 100,000 or 50,000 platelets per microlitre of the matrix forming agents.

For the preparation of the tissue generating product it is possible to obtain from a single blood sample, preferably obtained from the blood patient an autologous platelet rich plasma matrix suitable for a first application and from the remaining material sample, an autologous platelet poor plasma matrix suitable for a second application of the tissue generating product according to the invention.

According to the type of application, the tissue product could also comprise other elements, such as an effective amount of calcium containing compound dispersed in said matrix (for inducing the formation of a hard tissue, such as bone).

Said calcium phosphate containing compound is selecting from the group consisting of synthetic hydroxyapatite, CaCl₂, β-tricalcium phosphate, bone particles (denatured bone), bone particles (not denatured bone), apatite, aspidine, calcium sulfate, calcium carbonate, hydroxyapatite, (from coral reef), calcium gluconolactate, calcium gluconate, calcium lactate, calcium glutonate and mixtures thereof. (Preferably, said compound having no sharp or pointed edges).

Said effective amount of calcium containing compound could be inorganic particles containing calcium phosphate and having a mean particle size lower than 750 μm.

In the tissue bone generating product, the inorganic particles containing calcium phosphate have preferably a mean size comprised between 150 μm and 500 μm.

The bone particles comprised in the bone generating product according to the invention are preferably selected from the group consisting of craniofacial bone particles, iliac bone particles and mixture thereof. Said bone particles are preferably derivated from non denatured bones and the calcium phosphate containing particles have substantially no sharp and no pointed edge.

Advantageously, the bone particles have an average particle size comprised between 0.5 mm and 5 mm, preferably comprised between 0.5 and 3mm, most preferably about 1 mm (average in weight). The bone particles have for example the form of chips or flakes having an average particle size comprised between 0.5 mm and 5 mm, preferably comprised between 0.5 and 3 mm, most preferably about 1 mm (average in weight). According to a possible embodiment, the bone particles consist of a mixture of denatured bone particles (for example bone particles prepared by grinding a bone that has been treated by chemical(s), by irradiation, etc. for rendering it prion free.) and of not denatured bone particles. When using some denatured bone particles, the said particles of denatured bone can have a particle size lower than 0.5 mm, as said denatured bone particles are used to add some calcium to the product.

The tissue generating product of the invention comprises for example from 5% to 50% by volume of bone particles, advantageously from 10 to 40%, preferably from 20 to 30% by volume of bone particles. The bone particles forms preferably more than 90% by weight of the calcium containing compound present in the tissue generating product of the invention.

Furthermore, the tissue generating product according to the invention may also comprise further elements such as buffer agents, antibiotics, additives (selecting from the group consisting of growth factors, genes encoding growth factors, drugs, fatty acids, bactericides or virucides) and compounds for inducing the formation of matrixes and mixtures thereof.

Preferably, the said antibiotics have an anti-osteoclast effect.

Said additional element are preferably growth factor (PDGFAA,PDGFAB,PDGFBB, superfamily BTGF and family of BMP, such as BMP-1, etc.), gene coding BMP and/or BTGF, steric factors, calcium containing compounds, drugs, fatty acids, antibiotics or mixtures of antibiotics (preferably compound(s) having an anti-osteoclast effect, such as antibiotics of the tetracyclin group, Vibramycin®, Doxycycline®, Minocycline, Minocin® (Wyeth-Lederlee), and mixtures of compound(s) having an anti-osteoclast effect with another antibiotic(s), such as macrolide, penicillin based compounds, etc.), bactericide, virucide, fibrinogen, compounds inducing the formation of a matrix, buffer, zwitterionic buffer system at physiological pH, etc. and mixtures of said compounds or additives.

According to a detail of a preferred embodiment, the tissue generating product contains from 0.001 to 10% by weight antibiotic or antibiotics (calculated in its dry form), advantageously from 0.01% to 5% by weight, preferably from 0.02 to 1%, for example from 0.05 to 0.4% by weight. The antibiotic is advantageously selected from the group consisting of antibiotics having an anti-osteoclast effect (more specifically antibiotics of the tetracyclin group, Vibramycin®, Doxycycline®, Minocycline, Minocin® (Wyeth-Lederlee)), mixtures of antibiotics having an anti-osteoclast effect, and mixtures of one or more antibiotics having an anti-osteoclast effect with one or more other antibiotic(s) (preferably macrolide, penicillin based compounds, etc. and mixtures thereof).

Before its gelling, the tissue generating product has advantageously a pH substantially equal to the physiological pH, for example a pH comprised between 6.5 and 8, preferably about 7-7.5, pH measured at 37° C.

Among the recombinant growth factors present in the composition according to the invention, one may select (recombinant, which means that said growth factors do not present contaminants) tissue factors having no membrane binding sequence or having only an extra-cellular domain.

Preferably, said growth factors are selected from the group consisting of the human (recombinant) tissue factor (rhTF), the human (recombinant) platelet-derived growth factor (rhPDGF), the human (recombinant) transforming growth factor (rhTGF), the human (recombinant) insulin-like growth factor (rhIGF), the human (recombinant) epidermal growth factor (rhEGF), the human (recombinant) hepatocyte growth factor (rhHGF), . . . .

According to a preferred embodiment, the recombinant compound for generating thrombin (or tissue factor) is combined, preferably mixed, with at least a phospholipid selected from the group consisting of phosphatidylserine, derivatives thereof, phosphatidylserine having at least one fatty acid side chain, phosphatidylcholine, derivatives thereof, phosphatidylcholine having at least one fatty acid side chain, and mixtures thereof. Preferably, the recombinant compound for generating thrombin (or the tissue factor) is combined with at least a phospholipid selected from the group consisting of phosphatidylserine, derivatives thereof, phosphatidylserine having at least one fatty acid side chain, phosphatidylcholine, derivatives thereof, phosphatidylcholine having at least one fatty acid side chain, and mixtures thereof, the fatty acid side chain being selected from the group consisting of fatty acid chains with at least one double bond and with 6 to 24 carbon atoms, most preferably with 16 to 18 carbon atoms.

The tissue generating product according to the invention could also be a sealant which comprises said mentioned plasma matrix, one or more of said mentioned recombinant factor(s), (preferably a recombinant compound for generating thrombin) in presence of at least one of said mentioned phospholipid, preferably two different phospholipids and as a protein scatffold being the coagulation factor VII (FVII) which improves the sealant characteristics.

A further aspect of the present invention is related to a kit for the preparation of a sealant or a tissue generating product prepared by contacting said plasma matrix with a recombinant compound for generating thrombin (or growth factor), in presence of at least one phospholipid (preferably at least two different phospholipids), a protein scatffold for regeneration of said tissue or the coagulation factor VII and possibly an effective amount of calcium containing compound dispersed in said matrix for inducing the formation of the tissue being a bone (preferably said compound being inorganic particle containing a calcium phosphate having a mean particle size lower than 750 μm). Said kit comprising at least one system selecting from the group consisting of a vial containing, a recombinant compound for generating thrombin (or growth factor), the protein scatffold element or coagulation factor VII, and possibly an effective amount of calcium containing compounds for inducing the formation of the tissue being a bone (preferably inorganic particles containing calcium phosphate and having a mean particle size lower than 750 μm); two distinct vials, a first containing a recombinant compound for generating thrombin (or growth factor) while the second vial contains the protein scatffold element or the coagulation factor VII and possibly said effective amount of calcium containing compounds for inducing the formation of a tissue being a bone (preferably inorganic particles containing calcium phosphate and having a mean particle of size lower than 750 μm). The vial may also comprise the other elements present in the product according to the invention, such as a vial containing at least one buffer agent and at least one antibiotic and one or more of the other additives above mentioned.

The antibiotic formulation could be an oral antibiotic formulation, injectable antibiotic formulation, topic antibiotic formulation, spray antibiotic formulation and inhalated antibiotic formulation, said formulation being suitable for administrating to the patient an efficient dose of antibiotics at the place where the tissue has to be regenerated. Oral formulation and injectable formulation are preferred.

According to a detail of a preferred embodiment, the amount of antibiotic or antibiotics added to the sealant or tissue generating product or used during the coagulation of the matrix plasma tissue generating product or administered prior, during and/or after the application of the sealant or tissue generating product to the patient is such that the tissue generating product contains from 0.001 to 10% by weight antibiotic or antibiotics (calculated in its dry forms), advantageously from 0.01% to 5% by weight, preferably from 0.02 to 1%, for example from 0.05% to 0.4% by weight, more specifically from 0.2 to 0.3%. The antibiotic is advantageously selected from the group consisting of antibiotics having an anti-osteoclast effect (more specifically antibiotics of the tetracyclin group, Vibramycin®, Doxycycline®, Minocycline, Minocin® (Wyeth-Lederlee)), mixtures of antibiotics having an anti-osteoclast effect, and mixtures of one or more antibiotics having an anti-osteoclast effect with one or more other antibiotic(s) (preferably macrolide, penicillin based compounds, etc. and mixtures thereof).

Most preferably, at least one antibiotic is added to the mixture containing at least platelet rich plasma and calcium phosphate containing compound, before adding the recombinant compound that generates thrombin, but also advantageously when adding the recombinant compound that generates thrombin.

When no antibiotic is added to the mixture or when a low amount of antibiotic is added to the mixture, antibiotic(s) are advantageously given to the patient by oral administration, by injection, by topic application, by inhalation, preferably by oral administration and/or by injection (most preferably injection in the blood or percutaneous injection), prior and/or during and/or after the application of the bone generating product to the patient.

Preferably, the patient is submitted to at least one treatment with at least one antibiotic, said treatment being selected from the group consisting of:
oral administration of an efficient dose or effective amount of at least one antibiotic at least after the application of the sealant or tissue generating product to the patient;
oral administration of an efficient dose or effective amount of at least one antibiotic at least prior the application of the sealant or tissue generating product to the patient;
injection of an efficient dose or effective amount of at least one antibiotic at least after the application of the sealant or tissue generating product to the patient;
injection of an efficient dose of at least one antibiotic at least prior the application of the sealant or tissue generating product to the patient;
administration of an efficient dose or effective amount of at least one antibiotic at least for one day prior the application of the bone generating product and at least for one day after the application of the sealant or tissue generating product to the patient.

According to a specific embodiment, an effective amount of antibiotic is administered (most preferably orally or by injection) to the patient before the application of the sealant or tissue generating product of the invention, as well as after said application.

The invention relates also to a method for the preparation of the sealant or tissue generating product according to the invention in which :
a substantially homogeneous mixture is formed by mixing the plasma matrix (preferably platelet rich plasma or platelet poor plasma) with an effective amount of the protein scatffold or coagulation factor VII and possibly calcium containing compound for inducing the tissue generation when adding to the mixture of a growth factor such as a recombinant thrombin generating compound and at least one phospholipid,
the tissue factor or a recombinant thrombin generating compound and at least one phospholipid are added and mixed to the mixture of protein scatffold and coagulation factor VII and possibly calcium containing compound (such as bone particles or hydroxyapatite) and plasma matrix (preferably platelet rich plasma or platelet poor plasma), and
the said mixture is kept under conditions for ensuring a coagulation of the plasma matrix and the formation of the sealant or tissue generating product.

Preferably, the coagulation of the matrix is carried out in presence of oxygen and substantially without stirring. The said coagulation is most preferably carried out at a temperature comprised between 35° C. and 40° C., more specifically at a temperature of about 37° C.

Advantageously, at least two different phospholipids are added to the mixture, said addition being preferably carried out when adding the recombinant thrombin generating compound (or tissue factor).

In the process of the invention, the recombinant thrombin generating compound (or tissue factor) is advantageously combined with the phospholipid (above described), preferably with two phospholipids, the said compound combined with phospholipid(s) having advantageously the form of a lyophilized product, such as a lyophilized cake, powder or granules.

According to a detail of a preferred method of the invention, the coagulation of the plasma matrix is carried out in presence of at least one antibiotic or at least one antibiotic is added to the mixture after the coagulation of the matrix. The antibiotic or mixture of antibiotics can possibly be added to the other elements (the bone particles and/or the bone before its grinding and/or to the recombinant compound that generates thrombin or tissue factor and/or to a phospholipid).

In a preferred method of the invention, a gel (most preferably a hydrogel) is advantageously formed by the contact (preferably the mixing) of the plasma matrix with a recombinant compound for generating thrombin (or tissue factor) in presence of at least one phospholipid and the protein scatffold or the coagulation factor VII and possibly the inorganic particles, whereby during the gel formation, the pH of the plasma matrix is kept between 6 and 8.

The gel can also be formed in presence of at least one buffer agent. Possible buffers are for example TRIS buffer, solution of Ringe, sodium bicarbonate, and mixture thereof.

A last aspect of the present invention is related to a method for generating a tissue (such as bone) in a mammal patient (including the human) in need, said method comprising the step of applying at the place where the tissue (bone) has to be generated the sealant and/or tissue generating product according to the invention.

A last aspect of the present invention is also related to the use of the sealant and/or the tissue generating product according to the invention for the manufacture of a medicament in the treatment of tissue damages in a mammal patient (including the human).

The preferred characteristics of the various aspects of the present invention will be described in details in the enclosed examples.

### DESCRIPTION OF EXAMPLES

For the preparation of the said examples, the following products have been used:

PRP: platelet rich plasma of the patient to which a bone graft has to be placed. The platelet concentration of the plasma was 1,800,000 platelets per microliter of the plasma (higher concentration of platelets can also be used). The PRP was subjected to known usual treatments for the removal leucocyte, for obtaining a maximum proportion of living platelets, for bacteriological control, said PRP being active at least for 5 days. Prior its use, the PRP was shaken at a temperature of 37° C., the said shaking being achieved by shaking the container containing the PRP.

Thromboplastin: the thromboplastin used was a thromboplastin sold under the Trademark Innovin by the company DADE AG (Düdingen, Switzerland). The thromboplastin is a recombinant human tissue factor lyophilized combined with synthetic phospholipids, namely phosphatidylserine and phosphatidylcholine, said phospholipids having at least one fatty acid side chain, the fatty acid side chain being selected from the group consisting of fatty acid chains with at least one double bond and with 16-18 carbon atoms. Innovin is free of prothrombin, free of factor FVII, and free of factor FX. Calcium is present in Innovin. Innovin is a known product for diagnostic purposes. Innovin contains also some calcium, a zwitterionic buffer system at physiological pH. Innovin thromboplastin comprises a mixture of tissue factor and phospholipids, with a weight ratio tissue factor/phospholipids of about 1/300. (molar ratio TF/phospholipids of about 1/10,000).

Other thromboplastin can also be used, such as thromboplastin sold by American Diagnostica or thromboplastin developed by Henogen S A. The thromboplastin developed by Henogen S A comprises a tissue factor without membrane binding sequence and without intracellular domain. The Henogen tissue factor comprises thus only extra cellular domain. The Henogen tissue factor was expressed by yeast and recovered as soluble and glycosylated form in the culture medium. The tissue factor was purified by chromatography (in one or two steps, for example in two steps). The thromboplastin formulation of Henogen comprises also phospholipids, phosphatidyl-serine and phosphatidylcholine with a weight ratio phosphatidyl-serine/phosphatidylcholine of 3:7. The molar ratio TF/phospholipids is about 1/1000. The recombinant tissue factor is mixed with said phospholipids.

Bone particles: the bone particles have been prepared from iliac bones or craniofacial bone of the patient to whom a bone graft is needed. The fresh bone of the patient was ground in bone flakes (bone meal) having an average diameter of 1 mm. The bone particles are added to the PRP just after their preparation.

Water: water used is distilled, sterilized, pyrogen free water.

PepGen P-15®: this product sold by Friadent, Germany is a peptide enhanced by natural hydroxyapatite. The peptide has the formula GTPGPQGIAGQRGVV. Said peptide is bound to natural calcium phosphate particles (hydroxyapatite) with a size comprised between 250 μm and 420 μm. The particles are anorganic bone mineral heated at a temperature higher than 1100° C. The particles have been submitted to a rolling so as to remove sharp and pointed edges. The weight ratio peptide of formula GTPGPQGIAGQRGVV/calcium phosphate particle is about 250/1,000,000 or 0.00025. The particles are at least partly coated with said peptide.

PepGen P-15 Flow®: This product is similar to the product disclosed here above, except that it contains a resorbable gel matrix (made of sodium salt of a polycarboxymethyl ether of cellulose, glycerol and hydrogel).

### Example 1

In said example, 50 ml of PRP was placed in a sterilized container. A volume of 50 ml of PepGen P-15 or another protein scatfold above described was added to the PRP and mixed. The recipient is then heated under sterile conditions at 37.5° C. (for example by using a water bath having a temperature of 37.5° C., the said bath containing water and 0.9% NaCl), in an oxygen containing atmosphere.

10 mg Innovin was mixed with 2 ml distilled, sterile and pyrogen free water. The mixture water+Innovin was added to the PRP+PepGen P-15 mixture kept at a temperature of 37.5° C.

After about 10 minutes, without stirring, a gel is formed in the recipient, said gel being a bone generating product suitable for implant to the patient.

### Example 2

Example 1 has been repeated, except that 20 mg Innovin was mixed with 2 ml distilled, sterile and pyrogen free water, and was added to the mixture PRP+PepGen P-15.

### Examples 3 to 9

In said examples, example 1 was repeated except that the amount of reagents used was different.

| | Example | | | | | | |
|---|---|---|---|---|---|---|---|
| | 3 | 4 | 5 | 6 | 7 | 8 | 9 |
| PRP (ml) | 50 | 50 | 50 | 50 | 50 | 50 | 50 |
| PepGen | 35 | 35 | 45 | 40 | 60 | 75 | 75 |
| P-15 (ml) | | | | | | | |
| Innovin | 20 | 10 | 5 | 10 | 10 | 20 | 10 |
| (mg) | | | | | | | |
| Water (ml) | 2 | 2 | 2 | 2 | 2 | 4 | 4 |

### Examples 10 to 19

In said examples, example 1 was repeated except that the amount of reagents used was different.

| | Example | | | | | | | | | |
|---|---|---|---|---|---|---|---|---|---|---|
| | 10 | 11 | 12 | 13 | 14 | 15 | 16 | 17 | 18 | 19 |
| PRP (ml) | 50 | 50 | 50 | 50 | 50 | 50 | 50 | 50 | 50 | 50 |
| Bone | | | | 10 | 20 | 10 | 10 | 10 | 20 | 10 |
| particles | | | | | | | | | | |
| (ml) | | | | | | | | | | |
| cranio- | | | | | | | | | | |
| facial | | | | | | | | | | |
| Bone | 10 | 20 | 40 | 10 | 10 | 20 | 10 | 10 | 20 | 30 |
| particles | | | | | | | | | | |
| (ml) iliac | | | | | | | | | | |
| Innovin (mg) | 20 | 20 | 10 | 20 | 10 | 10 | 20 | 30 | 10 | 10 |
| PepGen P-15 | 35 | 40 | 50 | 50 | 50 | 60 | 65 | 70 | 75 | 75 |
| (ml) | | | | | | | | | | |
| Water (ml) | 2 | 2 | 4 | 2 | 2 | 2 | 2 | 2 | 4 | 4 |

The bone generating product of said examples 1 to 19 having the form of a gel can easily be implanted in a patient, for example in a human patient suffering a major maxillofacial atrophy. The bone generating product of the invention can easily be compacted in recesses of bones, and can be easily be shaped.

The bone generating product of the invention was used for volunteers suffering a major maxillofacial atrophy. Sinus lift grafts and on lay graft on the maxillofacial bone have been carried out. These tests have show a bone growth or the generation of bone where the bone generating product of the invention was applied.

### Example 20

A human bone was denatured and γ-irradiated so as to be prion free. The bone was ground in particles having an average (by weight) of 0.2 mm. After drying, log of bone particles were dry mixed with 10 mg dry Innovin, so as to obtain a mixture of recombinant compound for generating thrombin, phospholipid and a high level of calcium containing compound.

The so prepared mixture was then used for the preparation of a bone generating product of the invention.

The method of example 1 was repeated, except that the mixture 10 mg Innovin+10 g denatured bone particles was used instead of 10 mg Innovin alone, and except that a larger amount of sterile water was used (5-10 ml), amount water adjusted so as to prepare a paste.

### Example 21

Example 1 was repeated, except that before adding the recombinant thromboplastin, 200 μg Vibramycin® per ml mixture of PRP was added.

### Example 22

### Example 1 was repeated, except that before adding the recombinant thromboplastin, 100 μg Minocycline (Minocin®) per ml mixture of PRP was added.

### Example 23

Example 1 was repeated, except that before adding the recombinant thromboplastin, 50 μg Minocycline (Minocin®) per ml mixture of PRP was added.

### Example 24

Example 1 was repeated, except that before adding the recombinant thromboplastin, 20 μg Minocycline (Minocin®) per ml mixture of PRP was added.

### Example 25

Examples 1 to 24 have been repeated, except that PepGen P-15 Flow was used instead of PepGen P-15.

### Example 26

Examples 1 to 24 have been repeated except that Innovin was replaced by another recombinant tissue factor mixed with phospholipids. For example, the recombinant tissue factor is combined with at least a phospholipid selected from the group consisting of phosphatidylserine, derivatives thereof, phosphatidylserine having at least one fatty acid side chain, phosphatidylcholine, derivatives thereof, phosphatidylcholine having at least one fatty acid side chain, and mixtures thereof. Preferably, the recombinant compound for generating thrombin is combined with at least a phospholipid selected from the group consisting of phosphatidylserine, derivatives thereof, phosphatidylserine having at least one fatty acid side chain, phosphatidylcholine, derivatives thereof, phosphatidylcholine having at least one fatty acid side chain, and mixtures thereof, the fatty acid side chain being selected from the group consisting of fatty acid chains with at least one double bond and with 6 to 24 carbon atoms, most preferably with 16 to 18 carbon atoms.

According to a most preferred embodiment, the recombinant compound for generating thrombin is combined with a mixture of at least two phospholipids, a first phospholipid being selected from the group consisting of phosphatidylserine, derivatives thereof, phosphatidylserine having at least one fatty acid side chain, and mixtures therof, the fatty acid side chain of the phosphatidylserine having at least one fatty acid side chain being selected from the group consisting of fatty acid chains with at least one double bond and with 6 to 24 carbon atoms, preferably 16 to 18, while the second phospholipid is selected from the group consisting of phosphatidylcholine, derivatives thereof, phosphatidylcholine having at least one fatty acid side chain, and mixtures thereof, the fatty acid side chain of the phosphatidycholine having at least one fatty acid side chain being selected from the group consisting of fatty acid chains with at least one double bond and with 6 to 24 carbon atoms.

The weight ratio recombinant tissue factor/phospholipids is comprised for example between 1:500 and 1:50, such as in this example between 1:300 and 1:200, i.e. about 1:250.

In said examples, Innovin was replaced at the rate of 1.5 mg recombinant tissue factor+phospholipids per 10 mg Innovin used in examples 1 to 24.

Sealants were also prepared by mixing PRP, recombinant tissue factor of Henogen S A and phospholipids, with and without antibiotics.

1. A tissue-generating product comprising a plasma matrix, one or more growth factors, at least one phospholipid and a protein scaffold for the generation of said tissue wherein the protein scaffold is a matrix of collagen, reticuline and/or elastine fibers or a precursor thereof.

2. The tissue-generating product according to claim 1, wherein the precursor is tropocollagen or tropoelastine.

3. The tissue-generating product according to claim 1, wherein the plasma matrix is a coagulated matrix of platelet poor plasma comprising a platelet concentration lower than 500,000, 100,000 or 50,000 platelets per microlitre of the matrix forming agents.

4. The tissue-generating product according to claim 1, wherein the growth factor is selected from the group consisting of the human (recombinant) tissue factor (rhTF), the human (recombinant) platelet-derived growth factor (rhPDGF), the human (recombinant) transforming growth factor (rhTGF), the human (recombinant) insulin-like growth factor (rhIGF), the human (recombinant) epidermal growth factor (rhEG), and the human (recombinant) hepatocyte growth factor (rhHGF).

5. The tissue-generating product according to claim 1, which further comprises at least one buffer and at least one antibiotic.

6. The tissue-generating product according to claim 1, wherein the tissue is skin or an epithelial tissue of the stomach.

7. A kit for the preparation of a tissue-generating product according to claim 1, which contains a vial containing human growth factors, the protein scaffold element being selected from the group consisting of collagen, reticuline and/or elastine fibers and their precursors or two distinct vials, a first containing one or more growth factors, while the second vial containing a protein scaffold element selected from the group consisting of collagen, reticuline, and/or elastine fibers or their precursors, and optionally a last vial which may contain at least one buffered agent and at least one antibiotic.

8. A method for the preparation of a tissue-generating product according to claim 1, in which:
a substantially homogenous mixture is formed by mixing a plasma matrix with an effective amount of a protein scaffold element selected from the group consisting of collagen, reticuline and/or elastine fibers and their precursors;
a growth factor and at least one phopholipid are added and mixed to the mixture of the protein scaffold element and the plasma matrix, and
said mixture is kept under conditions for ensuring the coagulation of the plasma matrix and the formation of the tissue-generating product.

9. The method according to claim 8, wherein the coagulation of the matrix is carried out in the presence of oxygen and substantially without stirring.

10. The method according to claim 8, wherein the coagulation is carried out at a temperature comprised between 35° and 40° C.

11. (canceled)

12. A method for generating a tissue in a mammal patient, including a human in need thereof, said method comprising the step of applying at the place where the tissue has to be generated the tissue-generating product according to claim 1.

13. A method for treating tissue damage in a mammal patient, including human, comprising administering to the patient the tissue-generating product of claim 1.

14. The tissue-generating product according to claim 2, wherein the plasma matrix is a coagulated matrix of platelet poor plasma comprising a platelet concentration lower than 500,000, 100,000 or 50,000 platelets per microlitre of the matrix forming agents.

15. The tissue-generating product according to claim 2, wherein the growth factor is selected from the group consisting of the human (recombinant) tissue factor (rhTF), the human (recombinant) platelet-derived growth factor (rhPDGF), the human (recombinant) transforming growth factor (rhTGF), the human (recombinant) insulin-like growth factor (rhIGF), the human (recombinant) epidermal growth factor (rhEG), and the human (recombinant) hepatocyte growth factor (rhHGF).

16. The tissue-generating product according to claim 2, which further comprises at least one buffer and at least one antibiotic.

17. The tissue-generating product according to claim 2, wherein the tissue is skin or an epithelial tissue of the stomach.

18. A method for the preparation of a tissue-generating product according to claim 2, in which:
a substantially homogenous mixture is formed by mixing a plasma matrix with an effective amount of a protein scaffold element selected from the group consisting of collagen, reticuline and/or elastine fibers and their precursors;
a growth factor and at least one phopholipid are added and mixed to the mixture of the protein scaffold element and the plasma matrix, and
said mixture is kept under conditions for ensuring the coagulation of the plasma matrix and the formation of the tissue-generating product.

19. The method according to claim 18, wherein the coagulation of the matrix is carried out in the presence of oxygen and substantially without stirring.

20. The method of claim 8, wherein the coagulation is carried out at a temperature of about 37° C.

21. The method of claim 9, wherein the coagulation is carried out at a temperature of about 37° C.


SEQUENCE LISTING (1 sequence)

SEQ ID NO: 1 - protein, 15 aa, Artificial Sequence
  Synthesized
GTPGPQGIAGQRGVV